(19) United States
(12) Patent Application Publication
(10) Pub. No.: US 2017/0340223 A1
(43) Pub. Date: Nov. 30, 2017

(54) Implantable Automatic Wireless Intracranial Pressure Monitoring System and Method

(71) Applicant: ChuangHui Medical Technology Inc., Zhejiang (CN)

(72) Inventor: Dahong QIAN, Zhejiang (CN)

(21) Appl. No.: 15/169,488

(22) Filed: May 31, 2016

(51) Int. Cl. A61B 5/03; A61B 5/01; A61B 5/00

(52) U.S. Cl. CPC A61B 5/031; A61B 5/6868; A61B 5/746; A61B 5/002; A61B 5/01; A61B 5/0022; A61B 2560/0214

(57) ABSTRACT

A system for implantable automatic wireless intracranial pressure (ICP) monitoring is disclosed, including a control device and an implantable pressure transducer assembly. The implantable pressure transducer assembly is configured to be partially implanted in a skull of an individual being monitored, and the control device communicates and transfers data with the implantable pressure transducer assembly in a wireless manner. The system for implantable automatic wireless ICP monitoring can achieve easy and rapid ICP monitoring and entails a one-to-multiple mode in which multiple individuals can be simultaneously monitored by the single system based on wireless communications in WiFi, Bluetooth or a non-standard protocol.

### TECHNICAL FIELD

The present invention relates to a medical device and, in particular, to a system for implantable automatic wireless monitoring of intracranial pressure (ICP). The invention is also directed to a method for implantable automatic wireless ICP monitoring.

### BACKGROUND

Intracranial pressure (ICP) is the pressure inside the skull and reflects the pressure experienced by brain tissue. The body has various mechanisms by which it keeps the ICP stable, in particular, the control of cerebrospinal fluid (CSF) pressure through CSF production and adsorption. ICP is measured in millimeters of mercury (mmHg) and, at rest, is normally 7-15 mmHg for a supine adult, and shifts to a minus value (averagely −10 mmHg) when in a vertical body position. Changes in ICP are attributed to volume changes in one or more of the constituents contained in the cranium.

One of the most damaging aspects of brain trauma and other cranial conditions is an elevated intracranial pressure. An ICP increase (most commonly due to head injury leading to intracranial hematoma or cerebral edema), can crush brain tissue, shift brain structures, contribute to hydrocephalus, cause brain herniation, and restrict blood supply to the brain. In addition, it is also a possible cause of reflex bradycardia.

Elevated ICP can result in lowered cerebral perfusion pressure (CPP) and, if not controlled, further in vomiting, headache, blurred vision or loss of consciousness. A further increase in ICP can possibly cause permanent brain damage and eventually a fatal hemorrhage at the base of the skull. In adults, an ICP increase of about 20 mmHG is called pathologic intracranial hypertension (ICH) and considered as a medical/surgical emergency. Particular cases needing ICP monitoring include traumatic brain injury (TBI) victims, stroke victims, hydrocephalus patients, patients undergoing intracranial procedures, patients with brain tumors or the “shaken baby” syndrome, and patients treated with kidney dialysis or artificial liver support.

It is also possible for ICP to drop below normal levels, despite the fact that the elevation is more common and more severe. In both cases, the symptoms are often the same, leading many medical experts to believe that the symptoms are attributed to changes in the pressure rather that the pressure itself.

The existing ICP monitoring techniques are generally grouped into invasive and non-invasive, and the invasive group is further divided into those by invasion of soft tissues, for example, lumbar punctures, as well as those by cranial invasion. The cranial invasion techniques include three distinct ICP monitoring methods using respectively: an intraventricular catheter, which is a thin, flexible catheter threaded into one of the two lateral ventricles of the brain; a subarachnoid screw or bolt, disposed just through the skull in the space between the arachnoid and cerebral cortex; and an epidural transducer, placed into the epidural space beneath the skull.

In the non-invasive group, the accepted, commercially available ICP monitoring method includes: taking a CT, MRI, or other image of the head; and interpreting the image and observing changes in various features. This method requires a high level of skill to read and assess the images and requires that the patient be brought to the imaging equipment. In many cases, a scan is delayed or cancelled because the patient is not stable enough to be moved. Even after the patient is stable, the various tubes and equipment connections to the patient have to be accounted for during transport to the relevant imaging equipment, and as a result additional personnel may be required, with a consequent increase in cost. In addition, the scans themselves are single measurements-“snap-shots” in time, of which at least two are required to assess subtle changes and variations. A series of scans could approximate continuous monitoring, but is not economically practical.

Other existing non-invasive measurement methods have not found extensive use in clinical practice due to their drawbacks such as low accuracy and cumbersomeness. Therefore, it is the invasive methods that are currently popular in international clinical practice.

In a lumbar puncture or spinal tap, a clinician delicately passes a fine needle through the lower region of the back into the fluid of the spinal cord. Once the spinal spaces have been penetrated, ICP can be estimated by attaching a pressure transducer. The communication between the fluid in the spinal column and the cranium allows the physician to ascertain the pressure in the cranium responsive to CSF pressure. Though invasive, a lumbar puncture is sometimes preferred because it is a soft tissue procedure rather than a cranial procedure. Generally, a non-neuro clinician will feel uneasy when performing a cranial procedure, but will perform a lumbar puncture. This procedure does allow transient manipulation or sampling of the intracranial fluid system, but is often painful and many times results in after affects, and typically raises patient apprehension. Additionally, it is a short-term procedure and is generally not considered useful for long-term ICP monitoring.

Although the cranial invasive techniques are medically accepted and most routinely used, processes and devices used therein for ICP monitoring are far from perfection, in which one dedicated control device is respective to only one pressure transducer assembly (FIG. 1) having an interface terminal 1 inserted in the dedicated control device (generally bulky, not shown in FIG. 1 though). The pressure transducer assembly further includes a flexible tube 2 and a disposable implantable portion 3. An optical pressure transducer 4 is disposed at one end of the disposable implantable portion 3 and is configured to measure the ICP by using grating theory. Optical fibers 5 are further included for wired data transmission. Such a device is very expensive and, due to the one-to-one wired connections, inconvenient for use by a patient. Therefore, it is usually only used for the intensive care.

Thus, there is a long felt need for an easy-to-use device operative to provide a direct measurement of ICP with improved accuracy.

Chinese Patent Publication No. CN101744620A discloses a system for implantable automatic wireless ICP monitoring, including an intrasomatic part and an extrasomatic part. The extrasomatic part transmits a command to the intrasomatic part by means of radio electromagnetic waves, upon receipt of the command, the intrasomatic part detects ICP data using a piezoelectric transducer and feeds the ICP data back to the extrasomatic part via radio electromagnetic waves, thereby enabling automatic monitoring. The intrasomatic and extrasomatic parts of this system are fully independent of each other in terms of hardware, and rely on radio frequency (RF) communications for allowing automatic ICP monitoring when in use. However, the RF communications are passive, which leads to the following drawbacks: the intrasomatic part is not provided with a power supply and can thus not actively emit signals; in order for the communications to be enabled, the extrasomatic and intrasomatic parts have to be located close to each other; and the communications are carried out in a one-to-one mode.

With the progress in the wireless communication technology, further improvements in the existing ICP monitoring systems have become necessary.

### SUMMARY OF THE INVENTION

The technical problem to be solved by the present invention is to provide a system and a method for implantable automatic wireless intracranial pressure (ICP) monitoring, which enable easy and efficient ICP monitoring.

The above problem is solved by a system for implantable automatic wireless ICP monitoring according to the present invention, which includes a control device and an implantable pressure transducer assembly, wherein the implantable pressure transducer assembly is configured to be partially implanted in a skull of an individual being monitored, wherein the control device communicates and transfers data with the implantable pressure transducer assembly in a wireless manner, and wherein the implantable pressure transducer assembly includes: a wireless transmission control unit; a flexible tube in connection with the wireless transmission control unit; and an implantable section in connection with the flexible tube, the implantable section having a pressure transducer disposed at one end of the implantable section, the pressure transducer coupled to the wireless transmission control unit via a data cable, the data cable inserted within an internal lumen provided by the flexible tube and the implantable section.

Preferably, the wireless transmission control unit is disposed external to the skull and includes a first controller as well as a first power supply and a first wireless transmission module connected to the first controller.

Preferably, the control device includes a second controller as well as a second power supply, a data memory, control buttons, a display and a second wireless transmission module connected to the second controller.

Further, the first or second wireless transmission module may be a WiFi, Bluetooth or a non-standard wireless transmission module.

Preferably, the system for implantable automatic wireless ICP monitoring according to the present invention may include a plurality of implantable pressure transducer assemblies each configured to be partially implanted in a skull of a corresponding one of a plurality of individuals being monitored.

Preferably, the pressure transducer is a miniature high-precision pressure transducer having a diameter of less than 2 mm.

Preferably, the implantable pressure transducer assembly further includes a temperature transducer which is coupled to the wireless transmission control unit and disposed within the implantable section.

Also disclosed in this invention is a method for implantable automatic wireless ICP monitoring, using the system as defined above and including the steps of:

1) activating the control device and sequentially requesting and checking wireless signals from the implantable pressure transducer assemblies;

2) activating the implantable pressure transducer assemblies to obtain intracranial pressure measurements and transmit the intracranial pressure measurements as out-bound wireless signals;

3) determining by the control device whether the intracranial pressure measurements are normal and producing an alarm upon detection of an abnormal intracranial pressure measurement; and

4) determining whether to terminate the monitoring process, if yes, determinate the process, and if no, return to step 1).

Preferably, step 2) further includes obtaining intracranial temperature measurements.

Further, step 3) may include separately determining, by the control device, whether the intracranial pressures of the individuals being monitored exceed a normal range based on the intracranial pressure measurements transmitted from the implantable pressure transducer assemblies.

The system and method for implantable automatic wireless ICP monitoring according to the present invention have the following advantages:

1) Easy and Rapid Monitoring

Use of other (invasive) monitoring systems must be made in operating rooms and is limited to specialists. Additionally, the individual being monitored is not allowed to move freely due to the wired connections to the monitoring system.

In contrast, use of the present invention enables easy and rapid monitoring of the ICP of a patient based on wireless communications between the external control device and the wireless transmission control unit inside the implantable pressure transducer assembly which has been implanted in the skull of the patient by a minor surgical procedure. With this design, the monitoring can be accomplished in an automatic manner simply by manipulating several press buttons to activate automatic operation of the system, whenever and wherever as needed, for example, at home or in a ward at any time, without temporal or spatial limitations and without needing to be manipulated by a specialist.

2) High Accuracy

Use of the miniature high-precision pressure transducer for pressure measurements enables high-accuracy data transmission in the form of wireless digital signals.

3) High Applicability

The wireless communications with the external control device allows the individual being monitored to move in a rather large range, making it possible to be used in long-term continuous monitoring applications.

4) High Monitoring Efficiency

Significant improvements in monitoring efficiency can be achieved by using the single monitoring system to monitor multiple individuals based on communications between one control device and multiple pressure transducer assemblies in a wireless communication protocol properly selected from WiFi, Bluetooth and a non-standard protocol.

### BRIEF DESCRIPTION OF THE DRAWINGS

The present invention will be described in greater detail below with reference to a description of several specific embodiments, which is to be read in conjunction with the following accompanying drawings, in which:

FIG. 1 is a schematic illustration of a pressure transducer assembly in an existing system for implantable automatic wireless ICP monitoring;

FIG. 2 is a schematic illustration of a pressure transducer assembly in a system for implantable automatic wireless ICP monitoring according to the present invention;

FIG. 3 shows a one-to-one configuration embodiment of the system for implantable automatic wireless ICP monitoring according to the present invention;

FIG. 4 shows a one-to-multiple configuration embodiment of the system for implantable automatic wireless ICP monitoring according to the present invention;

FIG. 5 is a schematic circuit diagram of a wireless transmission control unit according to the present invention; and

FIG. 6 is a schematic circuit diagram of a control device according to the present invention;

FIG. 7 is a flow chart graphically illustrating a method for implantable automatic wireless ICP monitoring according to the present invention.

### DETAILED DESCRIPTION

### Embodiment 1

As shown in FIG. 3, a system for implantable automatic wireless ICP monitoring according to the present invention includes a control device and an implantable pressure transducer assembly implanted in the skull of an individual being monitored. The control device communicates and transfers data with the implantable pressure transducer assembly in a wireless manner

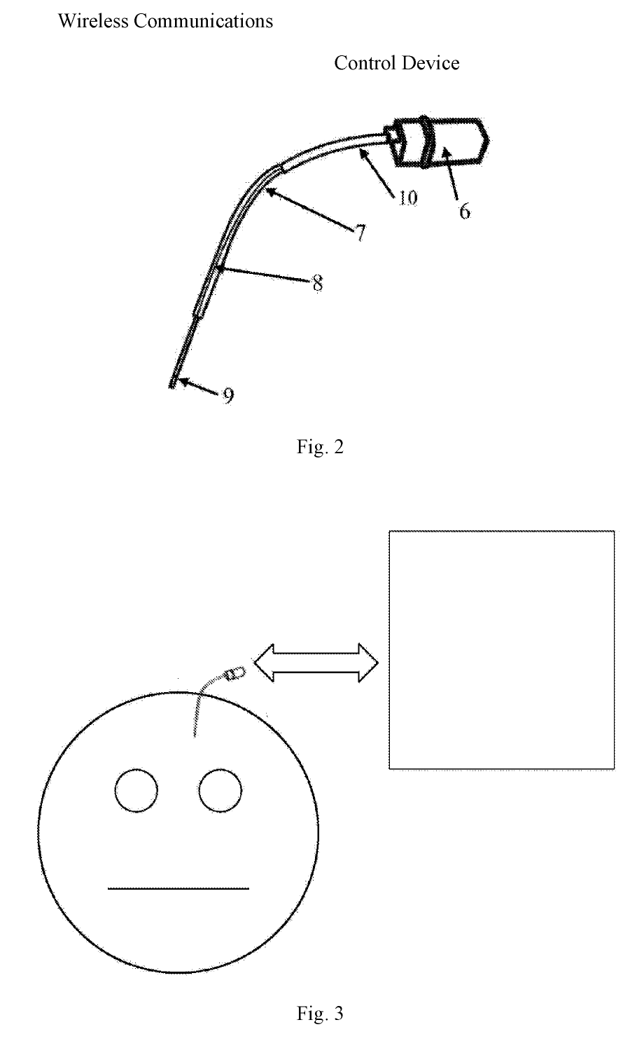

As shown in FIG. 2, the implantable pressure transducer assembly includes wireless transmission control unit 6, a flexible tube 10 in connection with the control unit 6, and an implantable section 7 connected to the flexible tube 10. A pressure transducer 9 disposed at one end of the implantable section 7 is coupled to the control unit 6 via a data cable 8, and the data cable 8 is inserted within an internal lumen provided by the flexible tube 10 and the implantable section 7.

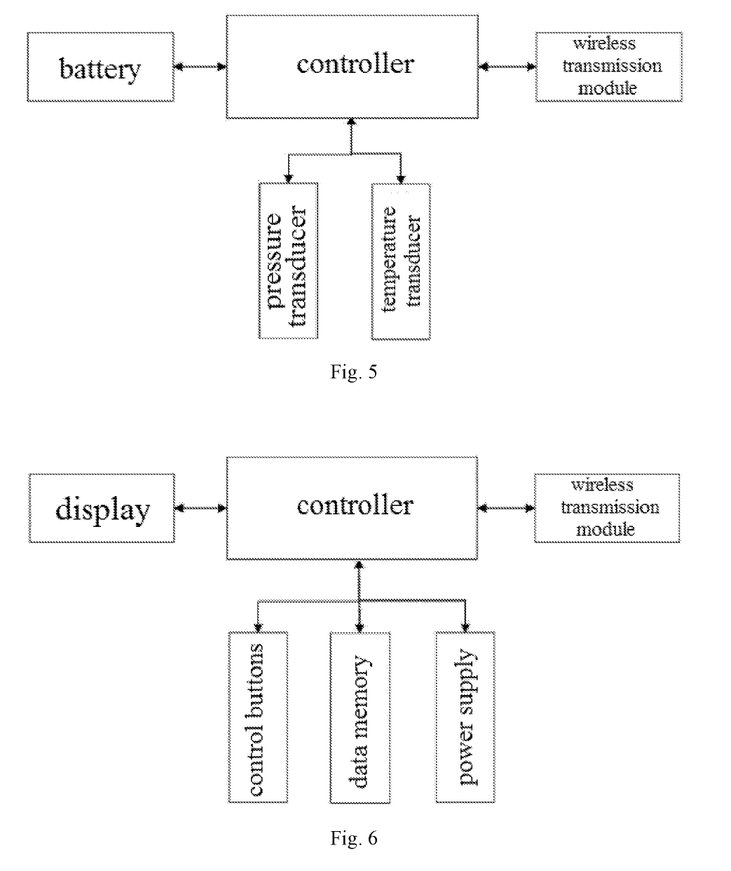

As shown in FIG. 5, in the system for implantable automatic wireless ICP monitoring according to the present invention, the wireless transmission control unit 6 is arranged external to the skull and incorporates a controller and, connected to the controller, a battery and a wireless transmission module.

As shown in FIG. 6, in the system for implantable automatic wireless ICP monitoring according to the present invention, the control device includes a controller and, connected to the controller, a power supply, a data memory, control buttons, a display and a wireless transmission module.

The wireless transmission modules of FIGS. 5 and 6 may be WiFi, Blue-tooth or a non-standard wireless transmission module.

Preferably, in the system for implantable automatic wireless ICP monitoring according to the present invention, the pressure transducer is a miniature high-precision pressure transducer having a diameter of less than 2 mm

Preferably, in the system for implantable automatic wireless ICP monitoring according to the present invention, the implantable pressure transducer assembly further includes a temperature transducer (as shown in FIG. 5) which is coupled to the wireless transmission control unit and disposed inside the implantable section.

### Embodiment 2

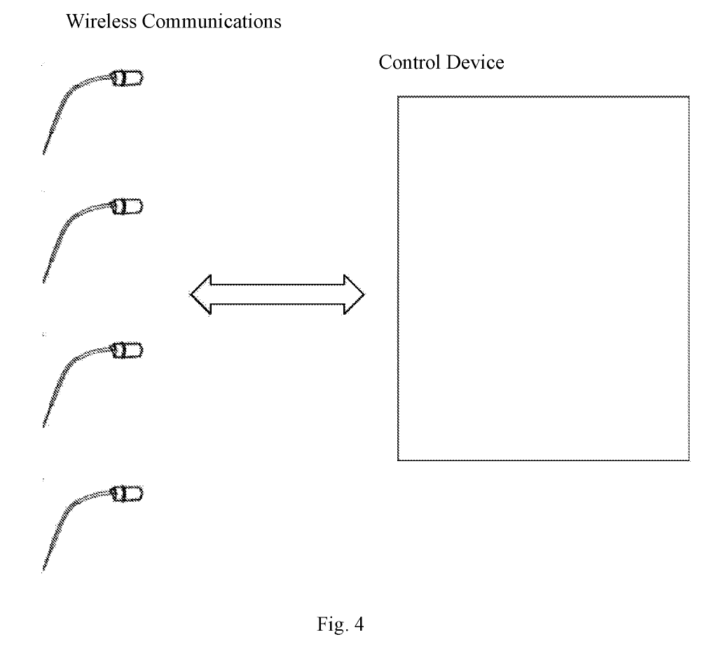

This Embodiment differs from Embodiment 1 only in that the system for implantable automatic wireless ICP monitoring according to Embodiment 2 of the present invention includes the control device and a plurality of implantable pressure transducer assemblies (as shown in FIG. 4) each implanted in the skull of a corresponding one of a plurality of individuals being monitored.

### Embodiment 3

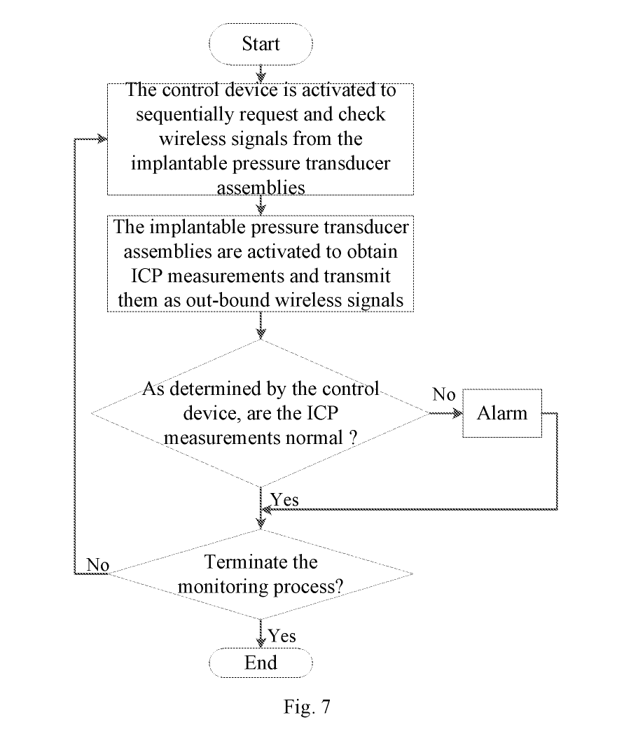

As shown in FIG. 7, a method for implantable automatic wireless ICP monitoring is also disclosed in the present invention, including the steps of:

1) activating the control device and sequentially requesting and checking wireless signals from the implantable pressure transducer assemblies;

2) activating the implantable pressure transducer assemblies to obtain ICP measurements and transmit them as out-bound wireless signals;

3) determining by the control device whether the ICP measurements are normal and producing an alarm upon detection of any abnormal measurement; and

4) determining whether to terminate the monitoring process, if yes, determinate the process, and if no, return to step 1).

Preferably, in the method for implantable automatic wireless ICP monitoring according to the present invention, step 2) further includes obtaining intracranial temperature measurements, and determining by the control device whether the ICP measurements are out of an normal range in step 3) refers to that the control device separately or sequentially determines whether the ICPs of the individuals exceed the normal range based on the ICP measurements transmitted from the implantable pressure transducer assemblies.

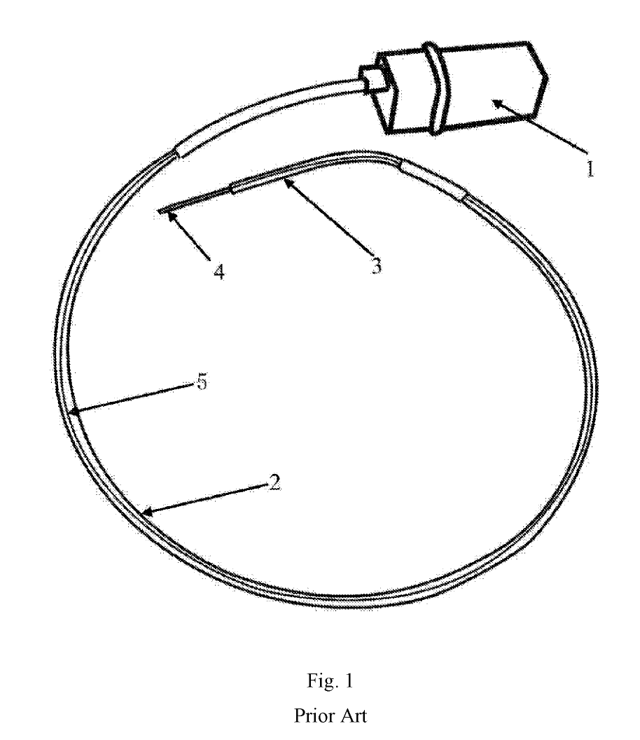

Despite their similar shapes, the implantable pressure transducer assembly according to the present invention have the following significant differences from the existing product shown in FIG. 1:

1) the transducer according to the present invention is a miniature pressure transducer, which operate differently from the optical pressure transducer in the exiting product, and produces measurements in the form of digital signals transmitted in a data cable, which are more sensitive and more accurate than the analog signals transmitted in the optical fibers of the exiting product;

2) the implantable pressure transducer assembly according to the present invention has a smaller overall size and uses a very short flexible tube and wireless transmission control unit which communicates wirelessly and does not need to be inserted in the control device, allowing movements of the individual being monitored in an significantly expanded range; and

3) multiple implantable pressure transducer assemblies according to the present invention can be connected to the control device and communicate therewith in one of several suitable wireless communication protocols.

In addition, the system and method for implantable automatic wireless ICP monitoring according to the present invention offer the additional advantages as follows:

1) Easy and Rapid Monitoring

Use of other (invasive) monitoring systems must be made in operating rooms and is limited to specialists. Additionally, the individual being monitored is not allowed to move freely due to the wired connections to the monitoring system.

In contrast, use of the present invention enables easy and rapid monitoring of the ICP of a patient based on wireless communications between the external control device and the wireless transmission control unit inside the implantable pressure transducer assembly which has been implanted in the skull of the patient by a minor surgical procedure. With this design, the monitoring can be accomplished in an automatic manner simply by manipulating several press buttons to activate automatic operation of the system, whenever and wherever as needed, for example, at home or in a ward at any time, without temporal or spatial limitations and without needing to be manipulated by a specialist.

2) High Accuracy

Use of the miniature high-precision pressure transducer for pressure measurements enables high-accuracy data transmission in the form of wireless digital signals.

3) High Applicability

The wireless communications with the external control device allows the individual being monitored to move in a rather large range, making it possible to be used in long-term continuous monitoring applications.

4) High Monitoring Efficiency

Significant improvements in monitoring efficiency can be achieved by using the single monitoring system to monitor multiple individuals based on communications between one control device and multiple pressure transducer assemblies in a wireless communication protocol properly selected from WiFi, Bluetooth and a non-standard protocol.

The Embodiments described above are several preferred embodiments of the present invention which are not intended to limit the invention and are susceptible to various modifications and changes by those skilled in the art. It is therefore intended that any alternations, equivalent substitutions and improvements made within the spirit and scope of the present invention are all considered to fall within the scope of protection of the invention.

1. A system for implantable automatic wireless intracranial pressure monitoring, comprising a control device and a plurality of implantable pressure transducer assemblies each configured to be partially implanted in a skull of a corresponding one of a plurality of individuals being monitored, wherein:
the control device wirelessly communicates and transfers data with the plurality of implantable pressure transducer assemblies, and
each of the plurality of implantable pressure transducer assemblies comprises:
a wireless transmission control unit;
a flexible tube in connection with the wireless transmission control unit; and
an implantable section in connection with the flexible tube, the implantable section having a pressure transducer which produces measurements in the form of digital signals disposed at one end thereof, the pressure transducer coupled to the wireless transmission control unit via a data cable which carries said measurements in the form of digital signals, the data cable inserted within an internal lumen provided by the flexible tube and the implantable section.

2. The system according to claim 1, wherein the wireless transmission control unit is disposed external to the skull and comprises a first controller as well as a first power supply and a first wireless transmission module connected to the first controller.

3. The system according to claim 2, wherein the first wireless transmission module is a WiFi, or other wireless transmission module.

4. The system according to claim 1, wherein the control device comprises a second controller as well as a second power supply, a data memory, control buttons, a display and a second wireless transmission module connected to the second controller.

5. The system according to claim 4, wherein the second wireless transmission module is a WiFi, or other wireless transmission module.

6. (canceled)

7. The system according to claim 1, wherein the pressure transducer is a miniature high-precision pressure transducer having a diameter of smaller than 2 mm.

8. The system according to claim 1, wherein each of the plurality of implantable pressure transducer assemblies further comprises a temperature transducer, the temperature transducer being coupled to the wireless transmission control unit and disposed within the implantable section.

9. A method for implantable automatic wireless intracranial pressure monitoring using the system as defined in claim 1, the method comprising the steps of:
1) activating the control device and sequentially requesting and checking wireless signals from the implantable pressure transducer assemblies;
2) activating the implantable pressure transducer assemblies to obtain intracranial pressure measurements and transmit the intracranial pressure measurements as out-bound wireless signals;
3) determining by the control device whether the intracranial pressure measurements are normal and producing an alarm upon detection of an abnormal intracranial pressure measurement; and
4) determining whether to terminate the monitoring process, if yes, terminate the monitoring process, and if no, return to step 1).

10. The method according to claim 9, wherein step 2) further comprises obtaining intracranial temperature measurements.

11. The method according to claim 9, wherein step 3) comprises separately determining, by the control device, whether intracranial pressures of the plurality of individuals being monitored exceed a normal range based on the intracranial pressure measurements transmitted from the implantable pressure transducer assemblies.
